(19) United States
(12) Patent Application Publication
(10) Pub. No.: US 2015/0044166 A1
(43) Pub. Date: Feb. 12, 2015

(54) COMPOSITIONS AND METHODS FOR TREATMENT OF EXERCISE-INDUCED PULMONARY HEMORRHAGE OR NASOPHARYNGEAL CICATRIX

(71) Applicant: Riddle Institute, Tomball, TX (US)

(72) Inventor: Michael Riddle, JR., Houston, TX (US)

(21) Appl. No.: 14/454,304

(22) Filed: Aug. 7, 2014

Related U.S. Application Data
(60) Provisional application No. 61/863,779, filed on Aug. 8, 2013.

(51) Int. Cl. C07K 14/435; C07K 14/485; A61M 11/00; C07K 14/54; C07K 14/475; C07K 14/52; C07K 14/50; C07K 14/535

(52) U.S. Cl. CPC C07K 14/435; C07K 14/50; C07K 14/485; C07K 14/535; C07K 14/54; C07K 14/503; C07K 14/475; C07K 14/5421; C07K 14/5431; C07K 14/5434; C07K 14/523; A61M 11/005; A61M 2250/00
USPC 424/85.2; 514/13.5; 514/21.2; 514/9.1; 514/9.6; 424/85.1; 514/9.5; 514/8.5; 514/8.1; 128/200.14; 128/200.16

(57) ABSTRACT

Methods, compositions, and medical device systems relating to treating exercise-induced pulmonary hemorrhage (EIPH) and nasopharyngeal cicatrix (NC) in a mammal. For example, one method comprises administering through inhalation a composition comprising a physiologically acceptable carrier and an effective amount of each of one or more stem cell derived factors. Exemplary stem cell derived factors include, but are not limited to, growth factors, chemokines, and cytokines. The mammal may be a horse, dog, camel, or Homo sapiens.

This application claims priority to U.S. Provisional Application No. 61/863,779 filed Aug. 8, 2013.

### BACKGROUND OF THE INVENTION

1. Field of the Invention

The present invention relates generally to the field of medical and veterinary treatment of exercise-induced pulmonary hemorrhage (EIPH) or nasopharyngeal cicatrix. More particularly, it concerns the use of compositions comprising effective amounts of one or more stem cell factors in the treatment of EIPH or nasopharyngeal cicatrix.

2. Description of Related Art

Exercise-induced pulmonary hemorrhage (EIPH) is a common condition among horses that race at high speeds, such as Thoroughbred and Standardbred race horses. The prevalence of EIPH in race horses is very high. Endoscopic examination of the trachea and bronchi demonstrate a prevalence of 43 to 75% (Pascoe et al., 1981a; Raphel and Soma, 1982; Mason et al., 1983). Evidence of hemosiderophages found in bronchial lavage specimens of race horses suggests that virtually all race horses experience some degree of EIPH.

In addition to horses, a number of other mammals engage in high-intensity physical activity which may lead to EIPH. Examples of such mammals include, but are not limited to, dogs, camels, and human beings.

Regardless of the species of mammal, EIPH causes decreased performance over time because of the pulmonary fibrosis that occurs during the healing process after an episode of EIPH. EIPH is progressive in nature and continues to worsen over time. EIPH is caused by the increased pulmonary capillary pressure during strenuous exercise events combined with the relatively excessive negative pressure generated in the small airways during rapid and deep breathing during strenuous exercise along with inflammation of the airway mucosa caused from the presence of blood. The pressure gradient created during strenuous exercise causes the blood to be pulled across the alveolar-capillary blood-gas barrier membranes into the small airways. When blood enters the small airways, it causes the endothelium of the small airways to become inflamed, which in turn causes the thin tissue membranes that separate the blood from the small airways to become more porous causing more blood to leak into the airways.

The current standard of care for EIPH in race horses is treatment with furosemide (marketed as Lasix by Sanofi S.A., Paris, France). Furosemide lowers the animal's blood pressure, increases the viscosity of its blood, and functions as a diuretic. However, as of 2013, several countries have banned the use of furosemide in the horse racing industry, and it may also be banned in horse racing in the United States in the near future.

Other patents disclose various treatments for EIPH. For example, Blackwater et al. U.S. Pat. Nos. 4,722,334 and 4,955,372, and Russell et al. U.S. Pat. No 6,027,713 are directed to the administration, via inhalation, of a humidified gas stream as a treatment for horses, including a treatment for EIPH.

However, there remains a need for compositions and methods for the treatment of EIPH.

Nasopharyngeal cicatrix (NC) is a condition that has been observed in horses in subtropical regions of the southern United States. The condition first presents as inflammation of the upper respiratory tract of the animal. Over time, the condition may progress to scarring of the upper respiratory tract, which may lead to narrowing of the animal's airways. In extreme cases, the horse may require a tracheostomy in order to breathe.

Risk factors for NC include the animal's age, pasturing of the animal, and summer season. The agent(s) responsible for NC are not yet known.

There exists a need for compositions and methods for the treatment of NC.

### SUMMARY OF THE INVENTION

In one embodiment, the present disclosure provides a method of treating a condition selected from exercise-induced pulmonary hemorrhage (EIPH) or nasopharyngeal cicatrix (NC) in a mammal, comprising: administering through inhalation a composition comprising a physiologically acceptable carrier and an effective amount of each of one or more stem cell derived factors.

In one embodiment, the present disclosure provides a composition, comprising: a physiologically acceptable carrier, and an amount of each of one or more stem cell derived factors, wherein the amount of each stem cell derived factor is effective to treat exercise-induced pulmonary hemorrhage (EIPH) or nasopharyngeal cicatrix (NC) in a mammal to whom the composition is administered through inhalation.

In one embodiment, the present disclosure provides a medical device system for administering through inhalation a composition to treat exercise-induced pulmonary hemorrhage (EIPH) or nasopharyngeal cicatrix (NC) in a mammal, comprising: a reservoir configured to store at least one dose of the composition; a nebulizer configured to nebulize said at least one dose of the composition; and a delivery device configured to receive the at least one nebulized dose of the composition from the nebulizer and to deliver the at least one nebulized dose of the composition to at least one of the oral cavity or the nasal cavity of the mammal.

### DESCRIPTION OF ILLUSTRATIVE EMBODIMENTS

The treatment modality described below demonstrates reduced airway inflammation and improved healing of the lung tissue after an episode of EIPH. The reduced inflammation decreases the amount of blood leaked into the airways. The improved healing from the treatment reduces the amount of lung fibrosis caused by the healing process after an episode of EIPH. Both of these mentioned desirable effects will allow racing mammals to remain competitive for longer periods of time because of the prevention of lung damage caused by EIPH.

The treatment modality described below also has been observed to reduce inflammation and cicatrix (scar) formation in the upper respiratory tract of a mammal presenting with NC. The reduced inflammation delays or reduces cicatrix formation. This may bring about reduced morbidity and/or mortality of a mammal, while allowing the mammal to be pastured, especially in summer months in subtropical regions of the southern United States.

In one embodiment, the present disclosure provides a method of treating a condition selected from exercise-induced pulmonary hemorrhage (EIPH) or nasopharyngeal cicatrix (NC) in a mammal, comprising: administering through inhalation a composition comprising a physiologically acceptable carrier and an effective amount of each of one or more stem cell derived factors.

“Treating” is used herein to refer to one or more of reducing airway inflammation during an episode of EIPH, improving healing of lung tissue after an episode of EIPH, or both. In the context of NC, “treating” is used herein to refer to one or more of reducing airway inflammation of a mammal presenting with NC, reducing scar formation in the respiratory tract of a mammal presenting with NC, or both. In either context, “improving healing” refers to accelerating healing, i.e., taking less time to reach a desired healthy end-state, and/or bringing the lung and/or upper respiratory tract tissue to a healthier final state. (All relative terms herein are in comparison to results seen when no treatment is administered).

By “stem cell derived factor” is meant a compound which is secreted by a stem cell and plays a role in cell signaling in one or more cell types. Examples of stem cell derived factors include, but are not limited to, growth factors, chemokines, and cytokines.

In one embodiment, the stem cell is of the same species as the mammal to which the composition is to be administered. By doing so, the risk of immune responses by the mammal to stem cell derived factors from another species may be reduced.

In one embodiment, each of said one or more stem cell derived factors is selected from the group consisting of adiponectin (Acrp30), Agouti-related peptide (AgRP), angiopoietin-2, basic fibroblast growth factor (bFGF), BTC, epidermal growth factor receptor (EGF-R), FAS, fibroblast growth factor (FGF)-4, FGF-9, granulocyte colony stimulating factor (G-CSF), glucocorticoid-induced tumor necrosis factor receptor (GITR), GITR-ligand, chemokine C—X—C motif ligand (GRO), hepatocyte growth factor (HGF), intercellular adhesion molecule (ICAM)-3, insulin-like growth factor (IGF)-1SR, IGF-binding protein (IGFBP)-3, IGFBP-6, interleukin-2 receptor alpha (IL-2Rα), interleukin-6 receptor (IL-6R), interleukin (IL)-8, IL-11, IL-12p40, IL-17, lymphotaktin, membrane cofactor protein (MCP)-1, macrophage migration inhibitory factor (MIF), macrophage inflammatory protein (MIP)-1α, MIP-1β, MIP-3β, macrophase stimulating protein (MSP) α, neurotrophin (NT)-4, oncostatin M, osteoprotegerin, phosphatidylinositol-glycan biosynthesis class F (PIGF), sgp130, soluble tumor necrosis factor receptor type 2 (sTNF RH), tissue inhibitor of metalloproteinase (TIMP)-1, TIMP-2, TNF-related apoptosis-inducing ligand (TRAIL) receptor 3 (R3), TRAIL R4, urokinase receptor (uPAR), vascular endothelial growth factor (VEGF), and VEGF-D.

In one embodiment, the composition further comprises one or more stem cells. In such an embodiment, one or more stem cell derived factor(s) of the composition may be secreted by the stem cell(s) before, during, or after administration of the compound.

By “effective amount” is meant an amount of a first stem cell derived factor of the composition which, in combination with an effective amount of each other stem cell derived factor of the composition, upon administration to the mammal via inhalation, reduces airway inflammation associated with EIPH, improves healing of lung tissue after an episode of EIPH, reduces airway inflammation of a mammal presenting with NC, reduces scar formation in the respiratory tract of a mammal presenting with NC, or two or more thereof “Effective amount” does not necessarily mean that the first stem cell derived factor alone has therapeutic efficacy.

Rather, it means the first stem cell derived factor, and all other stem cell derived factors of the composition, are each present in an amount that renders the composition as a whole therapeutically efficacious. The determination of effective amounts of one or more stem cell derived factors is a matter of routine experimentation for the person of ordinary skill in the art having the benefit of the present disclosure.

A quantity of the composition which contains effective amounts of one or more stem cell derived factors and is small enough to be delivered in a short time period (e.g. from 1 min to 30 min, such as from 5 min to 15 min), may herein be termed a “dose.”

The physiologically acceptable carrier may be any compound or mixture of compounds that does not react with the one or more stem cell derived factors, that allows inhalation of the composition, and that has no adverse effects on the mammal. Examples of physiologically acceptable carriers include, but are not limited to, water, saline, and buffers with pH and osmolality values that are roughly equivalent to the mammal's serum pH and osmolality, among others.

To facilitate inhalation, it may be desirable to administer the composition in a nebulized or aerosolized form. In one embodiment, the method further comprises nebulizing the composition by at least one of applying at least a partial vacuum to the composition or applying ultrasound to the composition, prior to administering the composition.

As stated above, administration of the composition is via inhalation. In one embodiment, the composition is delivered to one or more of the lungs, the bronchi, the trachea, the sinuses, the nasal mucosa, or the oral mucosa of the mammal. As will be apparent to the person of ordinary skill in the art, to deliver the composition to any of those locations, the composition must first be inhaled via at least one of the oral cavity (mouth) or nasal cavity (nose) of the mammal.

Administration may be performed at one or more times before, during, or after the onset of EIPH, or before, during, or after onset of NC. For example, if permissible under the rules of an organization overseeing a competition in which the mammal intends to take part, it may be desirable to administer at least one dose of the composition prior to the mammal's participation in the competition. Such a prior delivery may have a prophylactic benefit.

For another example, it may be desirable to administer one or more doses shortly after intense physical activity by the mammal. Administration in this context may be performed in response to observed symptoms of EIPH or related syndromes (e.g., nosebleed in race horses), but need not be. It may be desirable in this context to administer a first dose, observe one or more symptoms and/or physiological markers (e.g., hemosiderophage level in bronchial lavage samples), and administer a further dose if the observed symptoms and/or physiological markers indicate a lack of efficacy for the first dose.

For yet another example, it may be desirable to administer one or more doses after an acute episode of EIPH has ceased and while the mammal is healing from that acute episode. By doing so, pulmonary fibrosis that occurs during the healing process may be reduced. This may retard the progress of EIPH over time, thus allowing a mammal engaged in professional physical or athletic performance to extend its career. The precise number of doses administered and the frequency of administration during the healing process may be determined by the person of ordinary skill in the art as a matter of routine experimentation, in light of the present disclosure.

For another example, it may be desirable to administer one or more doses before, during, or after pasturing of the mammal, especially in summer months and/or in subtropical regions, such as subtropical regions of the United States. Administration in this context may be performed in response to observed symptoms of NC (e.g., nasopharyngeal inflammation in pastured horses), but need not be. It may be desirable in this context to administer a first dose, observe one or more symptoms and/or physiological markers, and administer a further dose if the observed symptoms and/or physiological markers indicate a lack of efficacy for the first dose.

Any delivery device configured to allow inhalation of the composition may be used. In one embodiment, administering comprises delivering the composition to the oral cavity of the mammal by an inhaler. “Inhaler” herein refers to a device with a mouthpiece configured for insertion into the mammal's mouth.

In one embodiment, administering comprises delivering the composition to at least one of the oral cavity and the nasal cavity of the mammal by a mask. In one embodiment, wherein the mammal is a horse, the mask may be a FLEXINEB™ equine nebulizer device (Nortev, Galway, Ireland).

Any mammal which may suffer EIPH or NC may be the subject of the method. In one embodiment, the mammal may be selected from the group consisting of non-human mammals and Homo sapiens. In a further embodiment, wherein the mammal is a non-human mammal, the non-human mammal may be selected from the group consisting of horses, dogs, and camels. In a particular embodiment, wherein the mammal may suffer and/or be at risk of NC, the mammal may be a horse.

In one embodiment, the present disclosure provides a composition, comprising: a physiologically acceptable carrier, and an amount of each of one or more stem cell derived factors, wherein the amount of each stem cell derived factor is effective to treat EIPH or NC in a mammal to whom the composition is administered through inhalation.

The physiologically acceptable carrier and the one or more stem cell derived factors may be as described above.

In one embodiment, the present disclosure provides a medical device system for administering through inhalation a composition to treat EIPH or NC in a mammal, comprising: a reservoir configured to store at least one dose of the composition; a nebulizer configured to nebulize said at least one dose of the composition; and a delivery device configured to receive the at least one nebulized dose of the composition from the nebulizer and to deliver the at least one nebulized dose of the composition to at least one of the oral cavity or the nasal cavity of the mammal.

The composition may be as described above.

Any device or component capable of nebulizing the composition may be used. In one embodiment, the nebulizer is selected from the group consisting of a pump and an ultrasound generator.

The delivery device may be an inhaler or a mask, such as those describe above. In one embodiment, the delivery device is further configured to conform to at least one of the mouth, the nose, or the snout of the mammal.

As described above, the mammal may be selected from the group consisting of non-human mammals and Homo sapiens. In embodiments wherein the mammal is a non-human mammal, the non-human mammal may be selected from the group consisting of horses, dogs, and camels.

The medical device system may be of any form, e.g., with separate reservoir, nebulizer, and delivery device, with such devices being modular and interconnectable, and/or with two or more of the reservoir, the nebulizer, and the delivery device contained in a single housing. For example, the FLEXINEB™ device referred to above comprises the reservoir, the nebulizer, and the delivery device contained in a single housing.

All of the compositions, methods, and apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions, methods, and apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method of treating a condition selected from exercise-induced pulmonary hemorrhage (EIPH) or nasopharyngeal cicatrix (NC) in a mammal, comprising:
administering through inhalation a composition comprising a physiologically acceptable carrier and an effective amount of each of one or more stem cell derived factors.

2. The method of claim 1, wherein at least one of the stem cell derived factors is selected from the group consisting of growth factors, chemokines, and cytokines.

3. The method of claim 1, wherein each of said one or more stem cell derived factors is selected from the group consisting of adiponectin (Acrp30), Agouti-related peptide (AgRP), angiopoietin-2, basic fibroblast growth factor (bFGF), BTC, epidermal growth factor receptor (EGF-R), FAS, fibroblast growth factor (FGF)-4, FGF-9, granulocyte colony stimulating factor (G-CSF), glucocorticoid-induced tumor necrosis factor receptor (GITR), GITR-ligand, chemokine C—X—C motif ligand (GRO), hepatocyte growth factor (HGF), intercellular adhesion molecule (ICAM)-3, insulin-like growth factor (IGF)-1SR, IGF-binding protein (IGFBP)-3, IGFBP-6, interleukin-2 receptor alpha (IL-2Rα), interleukin-6 receptor (IL-6R), interleukin (IL)-8, IL-11, IL-12p40, IL-17, lymphotaktin, membrane cofactor protein (MCP)-1, macrophage migration inhibitory factor (MIF), macrophage inflammatory protein (MIP)-1α, MIP-1β, MIP-3β, macrophage stimulating protein (MSP) α, neurotrophin (NT)-4, oncostatin M, osteoprotegerin, phosphatidylinositol-glycan biosynthesis class F (PIGF), sgp130, soluble tumor necrosis factor receptor type 2 (sTNF RII), tissue inhibitor of metalloproteinase (TIMP)-1, TIMP-2, TNF-related apoptosis-inducing ligand (TRAIL) receptor 3 (R3), TRAIL R4, urokinase receptor (uPAR), vascular endothelial growth factor (VEGF), and VEGF-D.

4. The method of claim 1, wherein the composition is delivered to one or more of the lungs, the bronchi, the trachea, the sinuses, the nasal mucosa, or the oral mucosa of the mammal.

5. The method of claim 1, wherein the mammal is selected from the group consisting of non-human mammals and Homo sapiens.

6. The method of claim 5, wherein the mammal is a non-human mammal, and the non-human mammal is selected from the group consisting of horses, dogs, and camels.

7. The method of claim 1, wherein administering comprises delivering the composition to the oral cavity of the mammal by an inhaler.

8. The method of claim 1, wherein administering comprises delivering the composition to at least one of the oral cavity and the nasal cavity of the mammal by a mask.

9. The method of claim 1, further comprising nebulizing the composition by at least one of applying at least a partial vacuum to the composition or applying ultrasound to the composition, prior to administering the composition.

10. The method of claim 1, wherein the condition is EIPH.

11. The method of claim 1, wherein the condition is NC.

12. A composition, comprising:
a physiologically acceptable carrier, and
an amount of each of one or more stem cell derived factors, wherein the amount of each stem cell derived factor is effective to treat exercise-induced pulmonary hemorrhage (EIPH) or nasopharyngeal cicatrix (NC) in a mammal to which the composition is administered through inhalation.

13. The composition of claim 12, wherein at least one of the stem cell derived factors is selected from the group consisting of growth factors, chemokines, and cytokines.

14. The composition of claim 12, wherein each of said one or more stem cell derived factors is selected from the group consisting of adiponectin (Acrp30), Agouti-related peptide (AgRP), angiopoietin-2, basic fibroblast growth factor (bFGF), BTC, epidermal growth factor receptor (EGF-R), FAS, fibroblast growth factor (FGF)-4, FGF-9, granulocyte colony stimulating factor (G-CSF), glucocorticoid-induced tumor necrosis factor receptor (GITR), GITR-ligand, chemokine C—X—C motif ligand (GRO), hepatocyte growth factor (HGF), intercellular adhesion molecule (ICAM)-3, insulin-like growth factor (IGF)-1SR, IGF-binding protein (IGFBP)-3, IGFBP-6, interleukin-2 receptor alpha (IL-2Rα), interleukin-6 receptor (IL-6R), interleukin (IL)-8, IL-11, IL-12p40, IL-17, lymphotaktin, membrane cofactor protein (MCP)-1, macrophage migration inhibitory factor (MIF), macrophage inflammatory protein (MIP)-1α, MIP-1β, MIP-3β, macrophase stimulating protein (MSP) α, neurotrophin (NT)-4, oncostatin M, osteoprotegerin, phosphatidylinositol-glycan biosynthesis class F (PIGF), sgp130, soluble tumor necrosis factor receptor type 2 (sTNF RII), tissue inhibitor of metalloproteinase (TIMP)-1, TIMP-2, TNF-related apoptosis-inducing ligand (TRAIL) receptor 3 (R3), TRAIL R4, urokinase receptor (uPAR), vascular endothelial growth factor (VEGF), and VEGF-D.

15. The composition of claim 12, wherein the amount of each stem cell derived factor is effective to treat EIPH.

16. The composition of claim 12, wherein the amount of each stem cell derived factor is effective to treat NC.

17. A medical device system for administering through inhalation a composition to treat exercise-induced pulmonary hemorrhage (EIPH) or nasopharyngeal cicatrix (NC) in a mammal, comprising:
a reservoir configured to store at least one dose of the composition;
a nebulizer configured to nebulize said at least one dose of the composition; and
a delivery device configured to receive the at least one nebulized dose of the composition from the nebulizer and to deliver the at least one nebulized dose of the composition to at least one of the oral cavity or the nasal cavity of the mammal.

18. The medical device system of claim 17, wherein the composition comprises a physiologically acceptable carrier, and an amount of each of one or more stem cell derived factors, wherein the amount of each stem cell derived factor is effective to treat EIPH or NC in the mammal.

19. The medical device system of claim 17, wherein the nebulizer is selected from the group consisting of a pump and an ultrasound generator.

20. The medical device system of claim 17, wherein the delivery device is further configured to conform to at least one of the mouth, the nose, or the snout of the mammal.

21. The medical device system of claim 17, wherein the mammal is selected from the group consisting of non-human mammals and Homo sapiens.

22. The medical device system of claim 21, wherein the mammal is a non-human mammal, and the non-human mammal is selected from the group consisting of horses, dogs, and camels.

23. The medical device system of claim 17, wherein the reservoir, the nebulizer, and the delivery device are contained in a single housing.
